(19) United States
(12) Patent Application Publication
(10) Pub. No.: US 2010/0292246 A1
(43) Pub. Date: Nov. 18, 2010

(54) FORMULATIONS OF 1-(4-BENZOYL-PIPERAZIN-1-YL)-2-[4-METHOXY-7-(3-METHYL-[1,2,4]TRIAZOL-1-YL)-1H-PYRROLO[2,3-C]PYRIDIN-3-YL]-ETHANE-1,2-DIONE

(75) Inventors: Chong-Hui Gu, North Brunswick, NJ (US); Qi Gao, Franklin Park, NJ (US); Shan-Ming Kuang, Florence, SC (US); Chiajen Lai, Kendall Park, NJ (US); Jaquan Kalani Levons, Ewing, NJ (US); Feng Qian, Hillsborough, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/847,014

(22) Filed: Jul. 30, 2010

Related U.S. Application Data
(63) Continuation of application No. 11/267,441, filed on Nov. 4, 2005, now abandoned.
(60) Provisional application No. 60/626,406, filed on Nov. 9, 2004.

(51) Int. Cl. A61K 31/496; A61P 31/18

(52) U.S. Cl. 514/253.04

(57) ABSTRACT

The instant invention provides formulations of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione; processes for the production of such formulations; and methods of treating HIV or AIDS with such crystalline materials or such formulations.

### CROSS-REFERENCE TO RELATED APPLICATIONS

This Continuation application claims the benefit of U.S. Ser. No. 11/267,441 filed Nov. 4, 2005, now abandoned, which in turn claims the benefit of U.S. Provisional Application Ser. No. 60/626,406 filed Nov. 9, 2004, now expired.

### FIELD OF THE INVENTION

The present invention relates to formulations of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione; processes for the production thereof; and methods of treating HIV and AIDS therewith.

### BACKGROUND OF THE INVENTION

HIV-1 (human immunodeficiency virus-1) infection remains a major medical problem, with an estimated 42 million people infected worldwide at the end of 2002. The number of cases of HIV and AIDS (acquired immunodeficiency syndrome) has risen rapidly. In 2002, ˜5.0 million new infections were reported, and 3.1 million people died from AIDS. Currently available drugs for the treatment of HIV include nine nucleoside reverse transcriptase (RT) inhibitors or approved single pill combinations (zidovudine or AZT (or Retrovir®), didanosine (or Videx®), stavudine (or Zerif®)), lamivudine (or 3TC or Epivir®), zalcitabine (or DDC or Hivid®), abacavir succinate (or Ziagen®), Tenofovir disoproxil fumarate salt (or Viread®), Combivir® (contains-3TC plus AZT), Trizivir® (contains abacavir, lamivudine, and zidovudine); three non-nucleoside reverse transcriptase inhibitors: nevirapine (or Viramune®), delavirdine (or Rescriptor®) and efavirenz (or Sustiva®), and eight peptidomimetic protease inhibitors or approved formulations: saquinavir, indinavir, ritonavir, nelfinavir, amprenavir, lopinavir, Kaletra (lopinavir and Ritonavir), and Atazanavir (Reyataz®). Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30 to 50% of patients ultimately fail combination drug therapies. Insufficient drug potency, non-compliance, restricted tissue penetration and drug-specific limitations within certain cell types (e.g. most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present (Larder and Kemp; Gulick; Kuritzkes; Morris-Jones et al; Schinazi et al; Vacca and Condra; Flexner; Berkhout and Ren et al; (Ref 6-14)). Therefore, novel anti-HIV agents exhibiting distinct resistance patterns, and favorable pharmacokinetic as well as safety profiles are needed to provide more treatment options.

U.S. patent application Ser. Nos. 10/038,306 (filed Jan. 2, 2002), 10/214,982 (filed Aug. 7, 2002), and 10/630,278 (filed Jul. 30, 2003) (all of which are herein incorporated by reference) disclose azaindoleoxoacetic piperazine derivatives and compositions that possess antiviral activity and are useful for the treatment of HIV and AIDS. U.S. patent application Ser. No. 10/630,278 discloses the compound I-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione, which has the chemical structure (I) (Compound (I)):

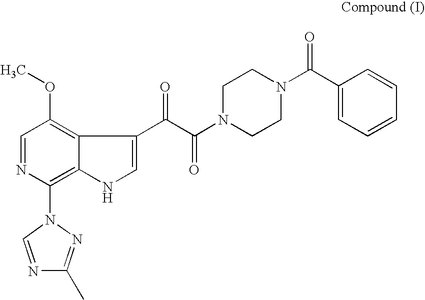

U.S. patent application Ser. No. 10/630,278 also discloses that Compound (I) can be prepared according to the following scheme:

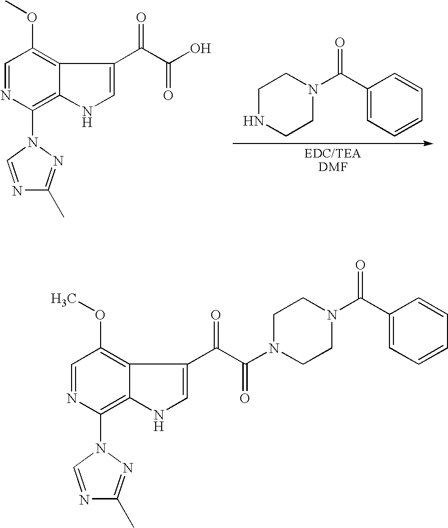

This reaction can also be performed by use of HATU and DMAP to provide more consistent yield of the title compound.

Co-pending application (Attorney Docket No. 10449-PSP, incorporated by reference herein in its entirety, entitled “CRYSTALLINE MATERIALS OF 1-(4-BENZOYL-PIPERAZIN-1-YL)-2-[4-METHOXY-7-(3-METHYL-[1,2,4]TRIAZOL-1-YL)-1H-PYRROLO[2,3-C]PYRIDIN-3-YL]-ETHANE-1,2-DIONE”) discloses various crystalline forms of the Compound (I). Its also discloses that the solubility of Compound (I) in crystalline form is typically low.

There exists a need to formulate Compound (I) effectively.

These and other aspects of the invention will become more apparent from the following detailed description.

### SUMMARY OF THE INVENTION

The present invention relates to several different formulations of Compound (I) (1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione). The present invention also relates to stable, reliable and reproducible methods for the manufacture, purification, and formulation of Compound (I) to permit its feasible commercialization. The present invention is directed to these, as well as other important aspects.

These and other aspects of the invention will become more apparent from the following detailed description.

### BRIEF DESCRIPTION OF THE FIGURES

The invention is illustrated by reference to the accompanying drawings described below.

FIG. 1(A). XRPD pattern of crystalline material Form P-1 of Compound (I).

FIG. 1(B). XRPD pattern of spray dried 40/60 Compound (I)/PVP-K-30.

### DETAILED DESCRIPTION OF THE INVENTION

Compound (I) exists in several different crystalline forms: P-1, P-2, P-3, and P-4. Of these four crystalline materials, P-1 is the most stable one, but it has an extremely low aqueous solubility of 0.0027 mg/mL. The present invention relates to formulations that effectively deliver Compound (I).

In a first embodiment, the present invention relates to formulating Compound (I) as a suspension of crystalline material P-1 in an aqueous solution.

In a second embodiment, the present invention relates to formulation Compound (I) as an amorphous powder.

An amorphous powder of Compound (I) can be obtained in a number of different ways, as would understand by one skilled in the art. Specifically, there are several different methods for obtaining such amorphous powder as follows:

The first method involves cooling the melt of crystalline P-1. The amorphous powder obtained has a glass transition temperature of about 140° C.

The second method involves forming a solution of Compound (I) and polyvinylpyrrolidone (PVP) in a solvent or solvent mixture, and then evaporating the solvent. The evaporation can be done, for example, through a Rotavapor™ or spray drying.

The formulations of the present invention may be administered to a patient in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They may be administered alone, but generally will be administered with a pharmaceutical carrier selected on the basis of the chosen route of administration and standard pharmaceutical practice.

The amount of Compound (I) in the present formulations, will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder. Obviously, several unit dosage forms may be administered at about the same time.

By way of general guidance, in the adult, suitable doses may range from about 0.001 to about 1000 mg/Kg body weight, and all combinations and subcombinations of ranges and specific doses therein. Preferred doses may be from about 0.01 to about 100 mg/kg body weight per day by inhalation, preferably 0.1 to 70, more preferably 0.5 to 20 mg/Kg body weight per day by oral administration, and from about 0.01 to about 50, preferably 0.01 to 10 mg/Kg body weight per day by intravenous administration. In each particular case, the doses may be determined in accordance with the factors distinctive to the subject to be treated, such as age, weight, general state of health and other characteristics which can influence the efficacy of the medicinal product.

For oral administration in solid form such as a tablet or capsule, these formulations of Compound (I) can be optional contain a non-toxic, pharmaceutically acceptable inert carrier, such as lactose, starch, sucrose, glucose, methylcellulose, magnesium stearate, dicalcium phosphate, calcium sulfate, mannitol, sorbitol and the like.

Preferably, in addition to the active ingredient, solid dosage forms may contain a number of additional ingredients referred to herein as “excipients”. These excipients include among others diluents, binders, lubricants, glidants and disintegrants. Coloring agents may also be incorporated. “Diluents”, as used herein, are agents which impart bulk to the formulation to make a tablet a practical size for compression. Examples of diluents are lactose and cellulose. “Binders”, as used herein, are agents used to impart cohesive qualities to the powered material to help ensure the tablet will remain intact after compression, as well as improving the free-flowing qualities of the powder. Examples of typical binders are lactose, starch and various sugars. “Lubricants”, as used herein, have several functions including preventing the adhesion of the tablets to the compression equipment and improving the flow of the granulation prior to compression or encapsulation. Lubricants are in most cases hydrophobic materials. Excessive use of lubricants is undesired, however, as it may result in a formulation with reduced disintegration and/or delayed dissolution of the drug substance. “Glidants”, as used herein, refer to substances which may improve the flow characteristics of the granulation material. Examples of glidants include talc and colloidal silicon dioxide. “Disintegrants”, as used herein, are substances or a mixture of substances added to a formulation to facilitate the breakup or disintegration of the solid dosage form after administration. Materials that may serve as disintegrants include starches, clays, celluloses, algins, gums and cross-linked polymers. A group of disintegrants referred to as “super-disintegrants” generally are used at a low level in the solid dosage form, typically 1% to 10% by weight relative to the total weight of the dosage unit. Croscarmelose, crospovidone and sodium starch glycolate represent examples of a cross-linked cellulose, a cross-linked polymer and a cross-linked starch, respectively. Sodium starch glycolate swells seven- to twelve-fold in less than 30 seconds effectively disintegrating the granulations that contain it.

The disintegrant preferably used in the present invention is selected from the group comprising modified starches, croscarmallose sodium, carboxymethylcellulose calcium and crospovidone. A more preferred disintegrant in the present invention is a modified starch such as sodium starch glycolate.

Preferred carriers include capsules or compressed tablets which contain the solid pharmaceutical dosage forms described herein. Preferred capsule or compressed tablet forms generally comprise a therapeutically effective amount of Compound (I) and one or more disintegrants in an amount greater than about 10% by weight relative to the total weight of the contents of the capsule or the total weight of the tablet.

Preferred capsule formulations may contain Compound (I) in an amount from about 5 to about 1000 mg per capsule. Preferred compressed tablet formulations contain Compound (I) in an amount from about 5 mg to about 800 mg per tablet.

More preferred formulations contain about 50 to about 200 mg per capsule or compressed tablet. Preferably, the capsule or compressed tablet pharmaceutical dosage form comprises a therapeutically effective amount of Form N-3 of Compound (I); a surfactant; a disintegrant; a binder; a lubricant; and optionally additional pharmaceutically acceptable excipients such as diluents, glidants and the like; wherein the disintegrant is selected from modified starches; croscarmallose sodium, carboxymethylcellulose calcium and crospovidone.

For oral administration in liquid form, Compound (I) can be combined with any oral, non-toxic pharmaceutically acceptable inert carrier such as ethanol, glycerol, water and the like. The liquid composition may contain a sweetening agent which to make the compositions more palatable. The sweetening agent can be selected from a sugar such as sucrose, mannitol, sorbitol, xylitol, lactose, etc. or a sugar substitute such as cyclamate, saccaharin, aspartame, etc. If sugar substitutes are selected as the sweetening agent the amount employed in the compositions of the invention will be substantially less than if sugars are employed. Taking this into account, the amount of sweetening agent may range from about 0.1 to about 50% by weight, and all combinations and subcombinations of ranges and specific amounts therein. Preferred amounts range from about 0.5 to about 30% by weight.

The more preferred sweetening agents are the sugars and particularly sucrose. The particle size of the powdered sucrose used has been found to have a significant influence in the physical appearance of the finished composition and its ultimate acceptance for taste. The preferred particle size of the sucrose component when used is in the range of from 200 to less than 325 mesh US Standard Screen, and all combinations and subcombinations of ranges and specific particle sizes therein.

Sterile injectable solutions may be prepared by incorporating Compound (I) in the required amounts, in the appropriate solvent, with various of the other ingredients enumerated herein, as required, followed by filtered sterilization. Generally, dispersions may be prepared by incorporating the sterilized active ingredient into a sterile vehicle which contains the dispersion medium and any other required ingredients. In the case of sterile powders for the preparation of sterile injectable solutions, the preferred methods of preparation may include vacuum drying and the freeze drying technique which may yield a powder of the active ingredient, plus any additional desired ingredient from the previously sterile-filtered solution thereof.

The liquid or suspension compositions may also contain other components routinely utilized in formulating pharmaceutical compositions. One example of such components is lecithin. Its use in compositions of the invention as an emulsifying agent in the range of from 0.05 to 1% by weight, and all combinations and subcombinations of ranges and specific amounts therein. More preferably, emulsifying agents may be employed in an amount of from about 0.1 to about 0.5% by weight. Other examples of components that may be used are antimicrobial preservatives, such as benzoic acid or parabens; suspending agents, such as colloidal silicon dioxide; antioxidants; topical oral anesthetics; flavoring agents; and colorants.

The selection of such optional components and their level of use in the compositions of the invention is within the level of skill in the art and will be even better appreciated from the working examples provided hereinafter.

Compound (I) may also be coupled with soluble polymers as targetable drug carriers. Such polymers can include polyvinylpyrrolidine pyran copolymer, polyhydroxypropylmethacrylamide-phenol, polyhydroxyethyl-aspartamidephenol or polyethylene oxide-polylysine substituted with palmitolyl residues. Gelatin capsules of Compound (I) may contain Compound (I) and the liquid or solid compositions described herein. Gelatin capsules may also contain powdered carriers such as lactose, starch, cellulose derivatives, magnesium stearate, stearic acid and the like. Similar diluents can be used to make compressed tablets. Both tablets and capsules can be manufactured as sustained release products to provide for continuous release of medication over a period of hours. Tablets can be sugar coated or film coated to mask any unpleasant taste and to protect the tablet from the atmosphere or enteric coated for selective disintegration in the gastrointestinal track.

In general, water, a suitable oil, saline, aqueous dextrose (glucose), and related sugar solutions and glycols, such as propylene glycol or polyethylene glycols are suitable carriers for parenteral solutions. Solutions for parenteral solutions are prepared by dissolving the crystalline Efavirenz in the carrier and, if necessary, adding buffering substances. Anti-oxidizing agents such as sodium bisulfate, sodium sulfite, or ascorbic acid either alone or combined, are suitable stabilizing agents. Citric acid and its salts and sodium EDTA may also be employed. Parenteral solutions may also contain preservatives, such as benzalkonium chloride, methyl- or propyl-paraben and chlorobutanol.

Suitable pharmaceutical carriers are described in Remington's Pharmaceutical Sciences, Mack Publishing Co., the disclosures of which are hereby incorporated herein by reference, in their entireties.

Pharmaceutical kits which may be useful for the treatment of various disorders, and which comprise a therapeutically effective amount of a pharmaceutical composition comprising a novel form of Compound (I) in one or more sterile containers, are also within the ambit of the present invention. The kits may further comprise conventional pharmaceutical kit components which will be readily apparent to those skilled in the art, once armed with the present disclosure. Sterilization of the container may be carried out using conventional sterilization methodology well known to those skilled in the art.

The present invention is further described in the following examples. All of the examples are actual examples. These examples are not to be construed as limiting the scope of the appended claims.

### EXAMPLES

### Example 1

### Heat-Cool-Heat DSC Experiment

Samples of Compound (I) was ramped from RT to 300° C. at 10° C./min in DSC 2920 cell at the atmosphere of N₂. The resulting molten liquid was air-cooled to RT to get a glassy solid, which was re-ramped from RT to 300° C. at 10° C./min in DSC 2920 cell.

### Example 2

### VT-XRPD Experiment

10.900 mg of Compound (I) was ramped from RT to 300° C. at 10° C./min in DSC 2920 cell at the atmosphere of N₂. The resulting molten liquid was air-cooled to RT to get a glassy solid, which was submitted for powder X-ray diffractometry (XRPD) data collection (28: 5-40° at 0.05°/step) at RT. This post XRPD sample was re-ramped from RT to 240° C. at 10° C./min in DSC 2920 cell to get a powder which was subjected to XRPD data collection at RT. Similarly, another sample of 10.9 mg of Compound (I) was ramped from RT to 100° C. at 10° C./min to get a powder which was sent for XRPD data collection at RT.

### Example 3

### Preparation of Amorphous Compound (I) by Spray Drying)

The following samples were prepared.

TABLE 1
Summary of research batches of spray dried intermediates

| | | | | Inlet/Outlet T(° C.); | | |
|---|---|---|---|---|---|---|
| | | | | Atomizing pressure | | |
| | Composition | Solvent | conc. | (Nl/hour); Pump rate | | |
| # | (w/w) | (v/v) | (w/v) | (%); Aspirator (%) | Yield | Notes |
| 1 | 40/60 | DCM* | 1.25% | 40/32; 500; 15; 100 | 60% | Amorphous by |
| | Compound (I)/ | | | | | XRPD/POM** |
| | PVP-K30 | | | | | |
| 2 | 40/60 | 70/30 | 2.5% | 100/65; 400; 15; 100 | 58% | Amorphous by |
| | Compound (I)/ | EtOH/H2O | | | | XRPD, partially |
| | PVP-VA | | | | | crystalline by POM |
| 3 | 40/60 | 70/30 | 2.5% | 100/65; 400; 15; 100 | 52% | Partially crystalline |
| | Compound (I)/ | EtOH/H2O | | | | by XRPD/POM |
| | PVP-K30 | | | | | |
| 4 | 40/60 | 70/30 | 1.25% | 100/57; 300; 30; 100 | 30% | Amorphous by |
| | Compound (I)/ | EtOH/H2O | | | | XRPD, partially |
| | PVP-K30 | | | | | crystalline by POM |
| 5 | 40/60 | 70/30 | 1.25% | 100/56; 400; 30; 100 | 43% | Amorphous by |
| | Compound (I)/ | EtOH/H2O | | | | XRPD, partially |
| | PVP-VA | | | | | crystalline by POM |
| 6 | 40/60 | DCM | 1.25% | 60/41; 400; 20; 100 | 65% | Amorphous by |
| | Compound (I)/ | | | | | XRPD/POM |
| | PVP-VA | | | | | |
| 7 | 40/55/5 | 70/30 | 1.25% | 90/55; 400; 20; 100 | 6.4% | Significant loss in |
| | Compound (I)/ | EtOH/H2O | | | | cyclone, low yield |
| | PVP-VA/ | | | | | |
| | Pluronic F127 | | | | | |
| 8 | 40/55/5 | 70/30 | 1.25% | 60/38; 400; 15; 100 | 13% | Significant loss in |
| | Compound (I)/ | EtOH/H2O | | | | cyclone; low yield |
| | PVP-VA/ | | | | | |
| | TPGS | | | | | |
| 9 | 40/60 | 19/80/1 | 3.25% | 100/60; 400; 30; 100 | 54% | Amorphous by |
| | Compound (I)/ | EtOH/DCM/ | | | | XRPD/POM; |
| | PVP-K30 | water | | | | significant loss in |
| | | | | | | drying chamber |
| 10 | 40/60 | 19/80/1 | 3.25% | 100/65; 400; 20; 100 | 40% | Amorphous by |
| | Compound (I)/ | EtOH/DCM/ | | | | XRPD/POM; |
| | PVP-K30 | water | | | | significant “beard |
| | | | | | | formation” |
| 11 | 40/60 | 19/80/1 | 3.25% | 80/52; 600; 30; 100 | 60% | Amorphous by |
| | Compound (I)/ | EtOH/DCM/ | | | | XRPD/POM; minor |
| | PVP-K30 | water | | | | “beard formation” |
| 12 | 40/59/1 | 19/80/1 | 6.25% | 60/44; 600; 30; 100 | 60% | Amorphous by |
| | Compound (I)/ | EtOH/DCM/ | | | | XRPD/POM |
| | PVP-K30/ | water | | | | |
| | TPGS | | | | | |
| 13 | 40/60 | 19/80/1 | 6.25% | 100/70; 700; 30; 100 | 72% | Amorphous by |
| | Compound (I)/ | EtOH/DCM/ | | | | XRPD/POM |
| | PVP-VA | water | | | | |
| 14 | 40/58/2 | 19/80/1 | 6.25% | 60/39; 700; 30; 100 | 74% | Amorphous by |
| | Compound (I)/ | EtOH/DCM/ | | | | XRPD/POM |
| | PVP-VA/ | water | | | | |
| | TPGS | | | | | |
| 15 | 40/60 | 20/80 | 6.25% | 60/42; 700; 30; 100 | 57% | Amorphous by |
| | Compound (I)/ | EtOH/DCM | | | | XRPD/POM |
| | PVP-K30 | | | | | |
| 16 | 40/60 | 20/80 | 6.25% | 100/70; 0.15 Mpa, | 65% | 70 g scale up in |
| | Compound (I)/ | EtOH/DCM | | 6 ml/min (Yamato) | | Yamato |
| | PVP-K30 | | | | | |
| 17 | 40/60 | 20/80 | 6.25% | 70/52; 700; 25; 100 | 65% | 90 g scale up in Buchi |
| | Compound (I)/ | EtOH/DCM | | | | B191 |
| | PVP-K30 | | | | | |

*DCM stands for dichloromethane
**XRPD and POM stands for powder X-ray diffractometry and polarized optical microscope, respectively.

### Example 4

### Spray Dried Formulation and Process I

1.3 g of Compound (I) and 1.95 g of PVP-K30 were dissolved in 100 ml of Jan. 19, 1980 (v/v) water/EtOH/DCM, total solid concentration: 3.25% w/v. The solution was filtered to remove extraneous matter. The filtered solution was sprayed at the rate of 30% (−15 mL/min) with atomizing nitrogen of 400N1/hour. The inlet temperature of the spray dryer was maintained at 100±5° C. The outlet temperature was maintained at 60±5° C. The resulting particles were separated in a cyclone and collected in a receiving vessel.

Range of processing conditions used in Buchi B-191 spray dryer:
Inlet temperature: 60-100° C.
Outlet temperature: 40-70° C.
Flow rate: ˜6-15 ml/min
Solution concentration: 3.25-6.25% w/v

### Example 5

### Spray Dried Formulation and Process II

16 g of Compound (I) and Plasdone-29/32 (equivalent to PVP K30) (24 g) are dissolved in a mixed solvent of 830.4 g DCM and 129.6 g EtOH (190 proof, containing 5% water). Total solid concentration is ˜4% w/w. The solution is sprayed through two-fluid nozzle (0.5 mm diameter) with atomizing nitrogen pressure at 0.5 bar and a liquid flow rate of ˜16 mL/min. The processing gas flow rate (hot nitrogen) is set at ˜25 kg/hr. The inlet temperature of the spray dryer is maintained at 70±5° C. The outlet temperature is maintained at 50±5° C. The resulting particles are separated in a cyclone and collected in a receiving vessel.

Additional conditions were tested using Niro's SDMicro spray dryer.
Range of processing conditions:
Inlet temperature: 48-102° C.
Outlet temperature: 31-91° C.
Flowrate: 5-20 mL/min
Solution concentration: 4-5% w/w

### Example 6

### Spray-Dried Formulation and Process III

Compound (I) (300 g) and PVP (Plasdone-29/32, 450 g) were dissolved in a pre-mixed solvent containing EtOH (200 proof), DCM, and H2O (2.98 kg/21.07 kg/0.20 kg). Total solid concentration is 3% w/w. The solution is sprayed in a Niro PSD-1 spray dryer equipped with a two-fluid nozzle (1.0 mm diameter). An in-line filter (Demicap Peplyn Plus, 5 microns opening) was used (before the solution is pumped to the spraying nozzle) to remove any particulates in the solution. The filtered solution was then sprayed through the two-fluid nozzle with atomizing nitrogen pressure at 0.8 bar. The processing gas flowrate (hot nitrogen) was set at ˜80 kg/hr. The inlet temperature of the spray dryer is maintained at 70±2° C. and outlet temperature was maintained at 45±2° C. Feed solution flowrate was adjusted accordingly (to maintain the processing temperatures) but was measured to be ca. 45 mL/min.

The resulting particles were separated in a cyclone and collected in a receiving vessel (A total of 0.324 kg SDI was collected). Additional material (0.195 kg) was collected from the bag filter which was located after the cyclone. Material was further oven-dried to remove residual solvent DCM.

Additional conditions were tested using Niro's SDMicro spray dryer.
Range of processing conditions:
Inlet temperature: 70-80° C.
Outlet temperature: 45-50° C.
Flowrate: 5-20 mL/min
Solution concentration: 3-4% w/w

### Example 7

### Spray Dried Formulation and Process IV

Compound (I) (434 g) and PVP (Plasdone-29/32, 651 g) were dissolved in a pre-mixed solvent containing EtOH (200 proof), DCM, and H2O (4.31 kg/30.49 kg/0.29 kg). Total solid concentration was 3% w/w. The solution was sprayed in a Niro PSD-1 spray dryer equipped with a two-fluid nozzle (1.0 mm diameter). An in-line filter (Demicap Peplyn Plus, 5 microns opening) was used (before the solution was pumped to the spraying nozzle) to remove any particulates in the solution. The filtered solution was then sprayed through the two-fluid nozzle with atomizing nitrogen pressure at 0.8 bar. The processing gas flowrate (hot nitrogen) was set at ˜80 kg/hr. The inlet temperature of the spray dryer was maintained at 70±2° C. and outlet temperature was maintained at 45±2° C. Feed solution flowrate was adjusted accordingly (to maintain the processing temperatures) but was measured to be ca. 45 mL/min.

The resulting particles were separated in a cyclone and collected in a receiving vessel. Material was further dried in a Niro-Aeromatic MP-1 Fluid Bed Processor to remove residual solvent.

### Example 8

### Testing Various Formulations in Dog Study

Four different samples were prepared and tested in dogs in oral exposure studies:
Sample A: spray-dried 40% Compound (I)/60% PVP K30 in capsule
Sample B: 5 mg/mL crystalline Compound (I) in 0.5% aqueous MC suspension, (D[4,3]=108.9 D50=31.7 D95=396.5)
Sample C: 20 mg/mL in 2% HPC/0.1% SLS (D95 188 nm)
Sample D: 10 mg/mL suspension in 90% PEG400/5% PVP/5% TPGS

The dosage is 200 mg Compound (I) per dog.

The results are listed in Table 2.

TABLE 2

| Sample | Cmax ± S.D. (ng/mL) | AUC ± S.D. (ng*h/mL) |
|---|---|---|
| A | 2497 ± 1245 | 19738 ± 7784 |
| B | 623 ± 298 | 3332 ± 235 |
| C | 2294 ± 1516 | 23936 ± 7647 |
| D | 3843 ± 1197 | 27642 ± 9354 |

### Example 9

### Testing Additional Formulations in Dog Study

Two different samples were prepared and tested in dogs in oral exposure studies:
Sample E: 5 mg/mL crystalline Compound (I) in 0.5% aqueous MC+0.1% SLS suspension (D[4,3]=20 D50=5 D90=50 micron)
Sample F: spray-dried 40% Compound (I)/60% PVP k30 in capsule Formulation

The dosage is 200 mg Compound (I) per dog.

The results are listed in Table 3.

TABLE 3

| Sample | AUC ± S.D. (ng*h/mL) |
|---|---|
| E | 2219 ± 865 |
| F | 11733 ± 8096 |

### Example 10

### Additional Capsule Formulation

Capsules of Compound (I) were prepared according to Table 4.

TABLE 4
Composition of Compound (I) Capsules

| | Reference | | Quantity per unit dose | | |
|---|---|---|---|---|---|
| Component | Standard | Function | 25 mg | 50 mg | 75 mg |
| Compound (I)/ | — | Active | 62.50 mg | 125.00 mg | 187.50 mg |
| Polyvinylpyrrolidone | | ingredient | | | |
| Spray Dried | | | | | |
| Intermediate^{a} | | | | | |
| Silicon dioxide | NF | Filler/Flow aid | 14.61 mg | 29.22 mg | 43.83 mg |
| Sodium Lauryl | NF | Dissolution | 0.63 mg | 1.25 mg | 1.88 mg |
| Sulfate | | Enhancer | | | |
| Magnesium Stearate | NF | Lubricant | 0.39 mg | 0.78 mg | 1.17 mg |
| Total weight | — | — | 78.13 mg | 156.25 mg | 234.38 mg |
| Capsules | — | — | Gray | Gray | Gray |
| | | | opaque #0 | opaque #0 | opaque |
| | | | capsule | capsule | #00 |
| | | | | | capsule |

^{a}The composition of Compound (I)/Polyvinylpyrrolidone Spray Dried Intermediate (40% w/w) is 40% Compound (I)/60% Polyvinylpyrrolidone (w/w). The function of polyvinylpyrrolidone is stabilizer of amorphous Compound (I).

What is claimed is:

1. A composition comprising 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione and polyvinylpyrrolidone.

2. The composition of claim 2, wherein the ratio of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione to polyvinylpyrrolidone is in the range from about 1:100 to about 100:1 (w/w).

3. The composition of claim 2, wherein the ratio of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione to polyvinylpyrrolidone is in the range from about 1:10 to about 10:1.

4. The composition of claim 3, wherein the ratio of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione to polyvinylpyrrolidone is about 4:6 (w/w).

5. The composition of claim 1, wherein the polyvinylpyrrolidone is polyvinylpyrrolidone K30.

6. The composition of claim 1, wherein the composition is amorphous.

7. An amorphous composition of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione prepared by the step comprising cooling a melt of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione.

8. An amorphous composition of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione prepared by the steps comprising of:
(a) preparing a solution of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione and polyvinylpyrrolidone or polyvinylpyrrolidone co-polymer in a solvent or solvent mixture selected from the group consisting of dichloromethane, mixture of dichloromethane/ethanol/water, and mixture of ethanol/water; and
(b) evaporating the solvent or solvent mixture.

9. The composition of claim 8, wherein step (b) is by spray-drying.

10. A method of preparing an amorphous composition of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione comprising the step of cooling a melt of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione.

11. A method of preparing an amorphous composition of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione comprising the steps of
(a) preparing a solution of 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione and polyvinylpyrrolidone or polyvinylpyrrolidone co-polymer in a solvent or solvent mixture selected from the group consisting of dichloromethane, mixture of dichloromethane/ethanol/water, and mixture of ethanol/water; and
(b) evaporating the solvent or solvent mixture.

12. The composition of claim 11, wherein step (b) is by spray-drying.

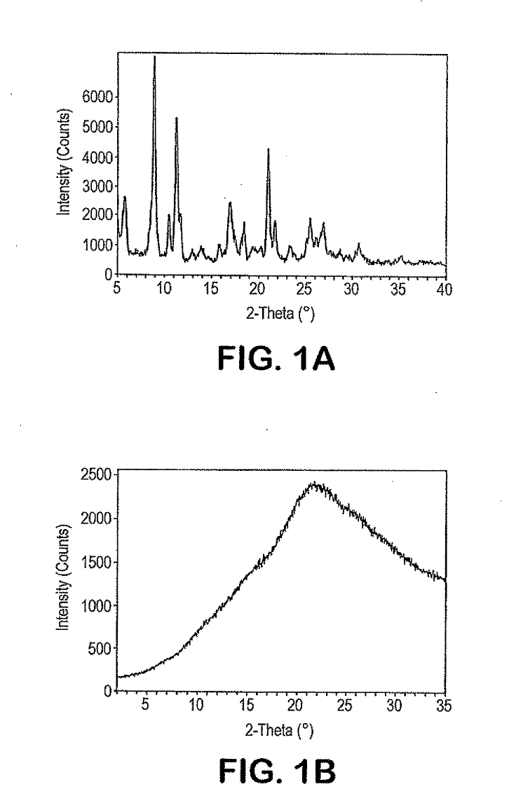

13. A composition comprising a suspension of
(1) Form I 1-(4-benzoyl-piperazin-1-yl)-2-[4-methoxy-7-(3-methyl-[1,2,4]triazol-1-yl)-1H-pyrrolo[2,3-c]pyridin-3-yl]-ethane-1,2-dione characterized by an X-ray powder diffraction pattern substantially in accordance with that shown in FIG. 1; and
(2) water.
